(19) United States
(12) Patent Application Publication
(10) Pub. No.: US 2004/0067263 A1
(43) Pub. Date: Apr. 8, 2004

(54) Method for preparating an anemone raddeana extract and the pharmaceutical composition containing the same as well as use thereof

(76) Inventors: Dayou Liu, Changchun City (CN); Datong Liu, Changchun City (CN); Jinwen Qiu, Changchun City (CN); Dafang Zhang, Changchun City (CN); Hongke Li, Changchun City (CN); Yifeng Liu, Changchun City (CN)

Correspondence Address: VENABLE, BAETJER, HOWARD AND CIVILETTI, LLP, P.O. BOX 34385, WASHINGTON, DC, 20043-9998, US

(21) Appl. No.: 10/466,287

(22) PCT Filed: Jan. 16, 2002
(86) PCT No.: PCT/CN02/00020

(30) Foreign Application Priority Data
Jan. 16, 2001 (CN) 01106086.7

(51) Int. Cl. A61K 35/78

(52) U.S. Cl. 424/725

(57) ABSTRACT

The present invention discloses a method for preparing Anemone Raddeana extract, comprising the following steps: a) pulverizing a rhizome, stem, leaf, flower, and fruit of Anemone Raddeana to obtain crude powders of Anemone Raddeana; b) extracting the crude powders of Anemone Raddeana with an ethanol solution, filtering, and recovering the ethanol; c) absorbing by a macroporous absorbing resin, removing pigments and saccharides, then eluting the resin until colorless with water or aqueous alkali solution, thus the fraction of saponin is retained in tile column; d) eluting with 50˜70% ethanol, recovering tile ethanol, adding gelatin, filtering precipitate, decoloring with decolor macroporous resin, eluting with 50˜70% ethanol, recovering the ethanol, concentrating and drying to obtain crude saponins of Anemone Raddeana. The present invention further relates to a method for extracting Raddeanin A. The method of the invention is convenient and suitable for industrial production. The Anemone Raddeana extract of the invention is formulated to pharmaceutical composition with pharmaceutically acceptable carriers. The pharmaceutical composition is used for treating malignant neoplasm diseases.

### TECHNICAL FIELD

The present invention relates to a process for extraction of effective constituent of Herbal medicine, particularly, a method for preparing Anemone Raddeana extract. The present invention further relates to a pharmaceutical composition containing Anemone Raddeana extract as well as use thereof.

### BACKGROUND ART

Anemone Raddeana (Liang Tou Jian) is dry rhizoma of Anemone Raddeana Regel (Ranunculaceae). It has been used for the treatment of chill arethitis, limb extremity, congle ache and carbuncle turgescense tanker in the field of traditional Chinese medicine due to its efficacy of relieving rheumatism decarbuncle and detumescence. It is demonstrated that the crude saponins, particularly Raddeanin A of Anemone Raddeana as well as the stem, leaf, flower, fruit thereof have anti-inflammation, easing pain, sedation, anti-bacterium activities in modern pharmacological studies. At present, the extraction and separation of Anemone Raddeana are still in the phase of laboratory study and can not be used for manufacturing and exploiting new drugs in industrial scale.

### DISCLOSURE OF THE INVENTION

It is an object of the present invention to provide a method for preparing Anemone Raddeana extract. The crude saponins as well as Raddeanin A with medical values are extracted from Anemone Raddeana. The method is convenient and suitable for industrial production.

It is another object of the present invention to provide a pharmaceutical composition containing Anemone Raddeana extract.

It is a further object of the present invention to provide a use of Anemone Raddeana extract.

The present invention provides a method for preparing Anemone Raddeana extract, comprising the following steps:

a) pulverizing a rhizome, stem, leaf, flower, and fruit of Anemone Raddeana to obtain crude powders of Anemone Raddeana;

b) extracting the crude powders of Anemone Raddeana with an ethanol solution, filtering, and recovering the ethanol;

c) absorbing by a macroporous absorbing resin, removing pigments and saccharides, then eluting the resin until colorless with water or aqueous alkali solution, thus the fraction of saponin is retained in the column;

d) eluting with 50˜70% ethanol, recovering the ethanol, adding gelatin, filtering precipitate, decoloring with decolor macroporous resin, eluting with 50˜70% ethanol, recovering the ethanol, concentrating and drying to obtain crude saponins of Anemone Raddeana.

In the step a) of the method of the invention, the rhizome, stem, leaf, flower, and fruit of Anemone Raddeana are pulverized and screened with 20 meshes to obtain crude powders.

In the step b), the crude powders of Anemone Raddeana are either extracted with 5-9 parts by weight of 50˜70% ethanol based on 1 part by weight of crude powders, or extracted by water decoction and alcohol precipitation, i.e. it is extracted by decoction with water, then is precipitated with alcohol.

In the step c), the aqueous alkali solution is, for example, aqueous sodium hydroxide solution.

The method of the present invention further comprises the following steps:

e) dissolving the crude saponins of Anemone Raddeana with MeOH, separating by silica gel dry column chromatography, eluting with CHCl₃:MeOH:H₂O (75˜55:45˜25:20˜1) to bottom of column;

f) collecting the silica gel in the column, eluting with MeOH until no saponin, recovering and distilling MeOH to dryness, and recrystallizing to obtain Raddeanin A.

In the step f) of the method of the invention, an alcohol is used for recrystallization.

The present invention further provides a pharmaceutical composition for treating cancer, comprising a therapeutically effective amount of Anemone Raddeana extract prepared by above methods and pharmaceutically acceptable carriers.

The present invention also provides a use of the Anemone Raddeana extract for the preparation of a medicament for the treatment of liver cancer, adenocarcinoma of stomach, lung cancer, and breast carcinoma.

The Anemone Raddeana extract prepared by the method of the invention may be made to any conventional pharmaceutically acceptable form.

The method of the invention is convenient and low cost, which is suitable for industrial production. The crude saponins of Anemone Raddeana and Raddeanin A prepared by the method of the invention have higher purity.

The Anemone Raddeana extract prepared by the method of the invention, as active substance, may be combined with one or more pharmaceutically acceptable carriers, thus make a pharmaceutical composition with any form.

For example, the dry power of crude saponins of Anemone Raddeana is mixed with starch, granulated with 70% ethanol, dried, and made into oral capsules.

For example, 10 parts by weight of Raddeanin A and 75 parts by weight of NaCl are dissolved in suitable amount of distilled water, adjust to pH7.5˜8.5 with 1-5% NaOH solution. Then 10000 ml of distilled water are added. After filtration, filled under aseptic conditions, lyophilized, and made into lyophilized powders for injection.

The extraction of the present invention can be made to a medicine for the treatment of cancers, particularly liver cancer, adenocarcinoma of stomach, lung cancer, and breast carcinoma.

The term “carrier” may be any conventional carrier in the field of pharmaceuticals, such as diluents, excipients, fillers, binders, wetting agents, disintegrating agents, sorbefacients, surfactants, absorbed carriers, and so on.

The Anemone Raddeana extract prepared by the method of the invention may be administrated in the form of oral, buccal, intravenous, intramuscular, or transdermal compositions. The composition may be made to tablets, granules, powders, capsules, suppositories, sprays, delaied-released agents, injections, and lyophilized powders for injection, and so on according to conventional methods. The extract may also mix with one or more other active ingredients and carriers to make desired dosage form.

The composition of the present invention preferably comprises 0.1%-99.5% by weight of active ingredients.

The dosages of the Anemone Raddeana extract may be determined depend on the mode of application and the age, weight, type of diseases, and severity of the patients. In general, dosages of about 0.01-20 mg/kg body weight per day come into consideration.

The therapeutically active substance of Anemone Raddeana is determined by extraction, separation and identifaction of Anemone raddeana. The inventor of the invention found the crude saponins of Anemone raddeana have significant anti-cancer activity. The rates of tumor inhibition are 79% and 71% for Hep-A-22 liver cancer of mouse and Lewis lung cancer of C₅₇ mouse respectively. The synthesis of DNA, RNA and protein of S₁₈₀ and ascites type liver cells of mouse are inhibited significantly in vitro. Furthermore, the rates of inhibition increase with the increasing of dosages and prolonging of time. The mechanism of anti-cancer is mainly due to inhibition of DNA synthesis of tumor cells.

This invention will be better understood from the following examples, which are for purposes of illustration and are not intended to limit the invention defined in the claims which follow thereafter.

### EXAMPLE

### Example 1

### Preparation of Crude Saponins of Anemone Raddeana

10 Kg of the rhizome, stem, leaf, flower, and fruit of Anemone Raddeana was pulverized and screened with 20 meshes. 50 Kg of 70% ethanol was added, refluxed under heating for 2 hrs, and repeated for three times. The combined extract was filtered, then ethanol was recovered. A gelatin was added, and resulted precipitation was filtered. The filtrate was absorbed by D₁₀₁ macroporous absorbing resin column. Washed the resin until colorless with water, then eluted with 70% ethanol until no saponin. Then recovered ethanol, sprayed drying, and obtained 0.5 kg of yellow-brown powder.

### Example 2

### Preparation of Crude Saponins of Anemone Raddeana

10 Kg of the rhizome, stem, leaf, flower, and fruit of Anemone Raddeana was pulverized and screened with 20 meshes. 50 Kg of 70% ethanol was added, refluxed under heating for 2 hrs, and repeated for three times. The combined extract was filtered, then ethanol was recovered. Sprayed drying to obtain powders, dissolved in 70% ethanol, and absorbed by D₁₀₁ macroporous absorbing resin column. Washed the resin until colorless with water, then eluted with 70% ethanol until no saponin. The eluant was decoloured by 1% active carbon for 30 mins, filtered immediately. Then recovered ethanol, sprayed drying, and obtained 0.5 kg of yellow-brown powder.

### Example 3

### Preparation of Crude Saponins of Anemone Raddeana

10 Kg of the rhizome, stem, leaf, flower, and fruit of Anemone Raddeana was pulverized and screened with 20 meshes. 100 Kg of water was added, decocted for 2 hrs, and repeated for three times. The combined extract was filtered, then the filtrate was concentrated to density of 1.18-1.20 (determined at 80° C.-85° C.). Ethanol was added to 70% concentration. Standing for 24 hrs, filtered. Ethanol was added to 90% concentration. Standing for 24 hrs, filtered, then ethanol was recovered. Sprayed drying and obtained 0.5 kg of yellow-brown powder.

### Example 4

### Preparation of Raddeanin A

100 g of crude powder prepared in Example 1 was diacolated with 500 mL of 70% ethanol, then ethanol was recovered. Dried under reduced pressure, the resulted residue was pulverized to powder. The powder was dissolved in methanol, and combined with five times silica gel. Separated by silica gel dry column chromatography, eluted with CHCl₃:MeOH:H₂O (75:25:5) to bottom of column. The silica gel column was taken out and cut into several pieces. Eluted with methanol, and identified with TLC. Fraction of Raddeanin A was collected, then eluted with methanol. Methanol was recovered under reduced pressure, recrystallized by ethanol, obtained white powdered crystal.

### Example 5

### Capsule Formulation

| | |
|---|---|
| crude saponins of Anemone Raddeana | 2500 g |
| Corn starch | 500 g |

Manufacturing Procedure: crude saponins of Anemone Raddeana and Corn starch were mixed well. Granulated with 70% ethanol, dried, and filled into capsules. 1000 capsules were prepared (0.3 g per capsule).

### Example 6

Lyophilized powders for injection formulation:

| | |
|---|---|
| Raddeanin A | 15 g |
| NaCl | 75 g |
| Injection water | 1000 ml |

Manufacturing Procedure: Raddeanin A and NaCl were dissolved in suitable amount of distilled water, adjust to pH8.0 with 1.5% NaOH solution. Then 1000 ml of distilled water are added. Filtrated through 0.2 μm microporous filter membrane, filled under aseptic conditions, and lyophilized.

### Example 7

### The Effect of Extracts Prepared from Example 1 on Inhibiting Growth of Tumor

In a study of pharmacodynamics, the capsule of crude saponins can inhibit tumour S₁₈₀, liver cancer, cervical neoplasms significantly, and the rates of tumor inhibition were 49%, 54%, 84% respectively. In proliferation test of extracorporeal cell strains, it was demonstrated that the growth of cell strains of the liver cancer, lung cancer, adenocarcinoma of stomach, and breast carcinoma were inhibited significantly under the direct action of the crude saponins, and the rate of inhibition were above 75%, 76%, 84% respectively.

### Example 8

The effect of Lyophilized powders of Raddeanin A for injection were detected as compared with 5-fluorouracil as control. The rates of tumor inhibition were 74% and 71% for Hep-A-22 liver cancer of mouse and Lewis lung cancer of C₅₇ mouse respectively. The rate of tumor inhibition was 54-41% for Bre-04 human breast carcinoma and C₀₁₋₀₆ human intestinal cancer. It was showed the obvious cytotoxicity for cells of liver tumor, lung tumor, breast tumor, and intestinal tumor, especially breast tumor was found in vitro test, and the IC₅₀ were lower than 100 μg/ml. The rates of tumor inhibition were 75%, 76%, 53%, 58% for human liver cancer, adenocarcinoma of stomach, lung cancer, and breast carcinoma respectively. The synthesis of S₁₈₀ cell ³H-TdR incorporated DNA was inhibited significantly by the injection of Raddeanin A.

1. A method for preparing Anemone Raddeana extract, comprising:
a) pulverizing a rhizome, stem, leaf, flower, and fruit of Anemone Raddeana to obtain crude powders of Anemone Raddeana;
b) extracting the crude powders of Anemone Raddeana with an ethanol solution, filtering, and recovering the ethanol;
c) absorbing by a macroporous absorbing resin, removing pigments and saccharides, then eluting the resin until colorless with water or aqueous alkali solution, thus the fraction of saponin is retained in the column;
d) eluting with 50˜70% ethanol, recovering the ethanol, adding gelatin, filtering precipitate, decoloring with decolor macroporous resin, eluting with 50˜70% ethanol, recovering the ethanol, concentrating and drying to obtain crude saponins of Anemone Raddeana.

2. The method according to claim 1, wherein the crude powders of Anemone Raddeana is extracted with 5-9 parts by weight of 50˜70% ethanol based on 1 part by weight of crude powders in the step b).

3. The method according to claim 1, wherein the crude powders of Anemone Raddeana is extracted by water decoction and alcohol precipitation in the step b).

4. The method according to claim 1, which further comprising:
e) dissolving the crude saponins of Anemone Raddeana with MeOH, separating by silica gel dry column chromatography, eluting with CHCl₃:MeOH:H₂O (75˜55:45˜25:20˜1) to bottom of column;
f) collecting the silica gel in the column, eluting with MeOH until no saponin, recovering and distilling MeOH to dryness, and recrystallizing to obtain Raddeanin A.

5. The method according to claim 1, wherein the recrystallization is carried out with an alcohol in the step f).

6. A pharmaceutical composition for treating cancer, comprising a therapeutically effective amount of Anemone Raddeana extract prepared by any methods according to claims 1-5 and pharmaceutically acceptable carriers.

7. A use of the Anemone Raddeana extract prepared by any methods according to claims 1-5 for the preparation of a medicament for the treatment of liver cancer, adenocarcinoma of stomach, lung cancer, and breast carcinoma.

8. Any pharmaceutically acceptable forms of the Anemone Raddeana extract prepared by any methods according to claims 1-5.
